SEQUENCE LISTING (9 sequences)

SEQ ID NO: 1 - protein, 367 aa, synthetic construct
LSHPLLGTALANGTGELSEHQQYVIGLFLSCLYTIFLFPIGFVGNILILVVNISFREKMTIPDLYFINLAVADLILVADSLIEVFNLHERYYDIAVLCTFMSLFLQVNMYSSVFFLTWMSFDRYIALARAMRCSLFRTKHHARLSCGLIWMASVSATLVPFTAVHLQHTDEACFCFADVREVQWLEVTLGFIVPFAIIGLCYSLIVRVLVRAHRMKKYTCTVCGYIYNPEDGDPDNGVNPGTDFKDIPDDWVCPLCGVGKDQFEEVEEHRGLRPRRQKALRMILAVVLVFFVCWLPENVFISVHLLQRTQPGAAPCKQSFRHAHPLTGHIVNLAAFSNSCLNPLIYSFLGETFRDKLRLYIEQKTNL

SEQ ID NO: 2 - DNA, 939 bp, synthetic construct
  source 1..939: mol_type = other DNA
ctgtcccacccgctcctgggcaccgccctggccaatgggacaggtgagctctcggagcaccagcagtacgtgatcggcctgttcctctcgtgcctctacaccatcttcctcttccccatcggctttgtgggcaacatcctgatcctggtggtgaacatcagcttccgcgagaagatgaccatccccgacctgtacttcatcaacctggcggtggcggacctcatcctggtggccgactccctcattgaggtgttcaacctgcacgagcggtactacgacatcgccgtcctgtgcaccttcatgtcgctcttcctgcaggtcaacatgtacagcagcgtcttcttcctcacctggatgagcttcgaccgctacatcgccctggccagggccatgcgctgcagcctgttccgcaccaagcaccacgcccggctgagctgtggcctcatctggatggcatccgtgtcagccacgctggtgcccttcaccgccgtgcacctgcagcacaccgacgaggcctgcttctgtttcgcggatgtccgggaggtgcagtggctcgaggtcacgctgggcttcatcgtgcccttcgccatcatcggcctgtgctactccctcattgtccgggtgctggtcagggcgcaccggcaccgtgggctgcggccccggcggcagaaggcgctccgcatgatcctcgcggtggtgctggtcttcttcgtctgctggctgccggagaacgtcttcatcagcgtgcacctcctgcagcggacgcagcctggggccgctccctgcaagcagtctttccgccatgcccaccccctcacgggccacattgtcaacctcgccgccttctccaacagctgcctaaaccccctcatctacagctttctcggggagaccttcagggacaagctgaggctgtacattgagcagaaaacaaatttg

SEQ ID NO: 3 - DNA, 1101 bp, synthetic construct
  source 1..1101: mol_type = other DNA
ctgtcccacccgctcctgggcaccgccctggccaatgggacaggtgagctctcggagcaccagcagtacgtgatcggcctgttcctctcgtgcctctacaccatcttcctcttccccatcggctttgtgggcaacatcctgatcctggtggtgaacatcagcttccgcgagaagatgaccatccccgacctgtacttcatcaacctggcggtggcggacctcatcctggtggccgactccctcattgaggtgttcaacctgcacgagcggtactacgacatcgccgtcctgtgcaccttcatgtcgctcttcctgcaggtcaacatgtacagcagcgtcttcttcctcacctggatgagcttcgaccgctacatcgccctggccagggccatgcgctgcagcctgttccgcaccaagcaccacgcccggctgagctgtggcctcatctggatggcatccgtgtcagccacgctggtgcccttcaccgccgtgcacctgcagcacaccgacgaggcctgcttctgtttcgcggatgtccgggaggtgcagtggctcgaggtcacgctgggcttcatcgtgcccttcgccatcatcggcctgtgctactccctcattgtccgggtgctggtcagggcgcaccggatgaagaagtatacctgtaccgtctgcggatacatttacaaccccgaggacggcgaccctgataacggcgtcaaccccggaactgacttcaaggatattcctgacgattgggtctgtcccctctgtggcgtcggcaaggaccaatttgaagaggtggaggaacaccgtgggctgcggccccggcggcagaaggcgctccgcatgatcctcgcggtggtgctggtcttcttcgtctgctggctgccggagaacgtcttcatcagcgtgcacctcctgcagcggacgcagcctggggccgctccctgcaagcagtctttccgccatgcccaccccctcacgggccacattgtcaacctcgccgccttctccaacagctgcctaaaccccctcatctacagctttctcggggagaccttcagggacaagctgaggctgtacattgagcagaaaacaaatttg

SEQ ID NO: 4 - DNA, 30 bp, synthetic construct
  source 1..30: mol_type = other DNA
attggcgcgccgctgtcccacccgctcctg

SEQ ID NO: 5 - DNA, 29 bp, synthetic construct
  source 1..29: mol_type = other DNA
attggccggcccggcaaatttgttttctg

SEQ ID NO: 6 - DNA, 30 bp, synthetic construct
  source 1..30: mol_type = other DNA
atgaagaagtatacctgtaccgtctgcgga

SEQ ID NO: 7 - DNA, 27 bp, synthetic construct
  source 1..27: mol_type = other DNA
ttcctccacctcttcaaattggtcctt

SEQ ID NO: 8 - DNA, 34 bp, synthetic construct
  source 1..34: mol_type = other DNA
tttgaagaggtggaggaacaccgtgggctgcggc

SEQ ID NO: 9 - DNA, 35 bp, synthetic construct
  source 1..35: mol_type = other DNA
tacaggtatacttcttcatccggtgcgccctgacc

(19) United States
(12) Patent Application Publication
(10) Pub. No.: US 2025/0042973 A1
(43) Pub. Date: Feb. 6, 2025

(54) RECOMBINANT PROTEIN OF GPER

(71) Applicant: Wuhan University, Wuhan (CN)

(72) Inventors: Jin XIANG, Wuhan (CN); Ying ZHAO, Wuhan (CN); Miaomiao QI, Wuhan (CN); Jing REN, Wuhan (CN); Yujie YAN, Wuhan (CN)

(73) Assignee: Wuhan University, Wuhan (CN)

(21) Appl. No.: 18/788,909

(22) Filed: Jul. 30, 2024

(30) Foreign Application Priority Data
Aug. 1, 2023 (CN) 2023109590395

(51) Int. Cl. C07K 14/72; C12N 15/63; G01N 33/535

(52) U.S. Cl. CPC C07K 14/723; C12N 15/63; G01N 33/535; C07K 2319/43

(57) ABSTRACT

Recombinant protein of GPER is provided. The recombinant protein has the biological activity of G protein-coupled estrogen receptor.

### CROSS-REFERENCE TO RELATED APPLICATIONS

The application claims priority to Chinese patent application No. 2023109590395, filed on Aug. 1, 2023, the entire contents of which are incorporated herein by reference.

### SEQUENCE LISTING

The sequence listing xmL file submitted herewith, named “Sequence_Listing.xmL”, created on Jun. 25, 2024, and having a file size of 11,264 bytes, is incorporated by reference herein.

### TECHNICAL FIELD

This disclosure relates to recombinant protein of GPER, specifically, the recombinant protein has the biological activity of G protein-coupled estrogen receptor.

### BACKGROUND

The statements herein provide background information relevant to the present disclosure only and do not necessarily constitute prior art.

G protein-coupled estrogen receptor (GPER) is a novel estrogen receptor with seven trans-membrane domains, which can bind to and be activated by estrogen, mediate the rapid response of estrogen in cells, and regulate the expression of genes. GPER is widely expressed in various tissues and is involved in the regulation of glucose lipid metabolism, pancreas, kidney, bone density, skin pigmentation, heart rate regulation, immune system and nervous system function.

GPER plays an important role in the occurrence and development of female reproductive cancers such as breast cancer, ovarian cancer and endometrial cancer. For example, activation of GPER can promote the proliferation, hypoxia tolerance and angiogenesis of triple-negative breast cancer cells. High GPER expression is associated with poor prognosis for breast and endometrial cancer, and high GPER expression is a favorable prognostic factor for Triple-Negative Breast Cancer (TNBC).

And, GPER is a novel anticancer drug target. In March 2021, a GPER agonist, LNS8801, was granted fast track designation by the US FDA for the treatment of patients with metastatic or unresectable melanoma whose disease has progressed after anti-PD-1 or anti-PD-L1 therapy, becoming the first GPER regulator drug to be used in the clinic.

In addition, GPER can also be used for drug screening, antibody preparation, and for the preparation of western blot kits, ELISA kits, etc. The dimer formed by GPER can be activated instantaneously due to its unique spatial structure. This is essential for studying the signaling mechanism of the GPER receptor and developing agonists that initiate GPER activation patterns.

### SUMMARY

In one aspect, embodiments disclose a recombinant protein of GPER. The recombinant protein shows as SEQ ID NO:1.

In another aspect, embodiments disclose a polymer. The polymer includes a plurality of a recombinant protein of GPER. The recombinant protein shows as SEQ ID NO:1. The plurality is selected from 2, 3, 4 or 5.

In another aspect, embodiments disclose a synthetic DNA. The synthetic DNA codes a recombinant protein of GPER. The recombinant protein shows as SEQ ID NO:1.

In another aspect, embodiments disclose a recombinant plasmid. The recombinant plasmid includes a basal vector and a synthetic DNA. The synthetic DNA codes a recombinant protein. The recombinant protein shows as SEQ ID NO:1.

In another aspect, embodiments disclose a recombinant host. The recombinant includes a host and a recombinant plasmid in the host. The recombinant plasmid includes a basal vector and a synthetic DNA. The synthetic DNA codes a recombinant protein of GPER. The recombinant protein shows as SEQ ID NO:1.

In another aspect, embodiments disclose uses of a recombinant protein of GPER. The recombinant protein shows as SEQ ID NO:1. The uses are selected from preparations of antibodies, drug screening kits, Western Blot kits, ELISA kits, or agonists for initiating the activation of GPER.

### BRIEF DESCRIPTION OF DRAWINGS

FIG. 1 shows the structure of the first plasmid provided with embodiments.

FIG. 2 shows the Agarose electrophoretic images of the first target sequence and the second target sequence provided with embodiments. Lane A refers to DNA ladder (Thermo Fisher, 10-250 kDa). Lane b refers to the first target sequence. Lane C refers to the second target sequence.

FIG. 3 shows the blue-white plaque plate of DH10Bac cells transformed by the second plasmid provided with embodiments. White spots marked by a red circle refer to positive colonies.

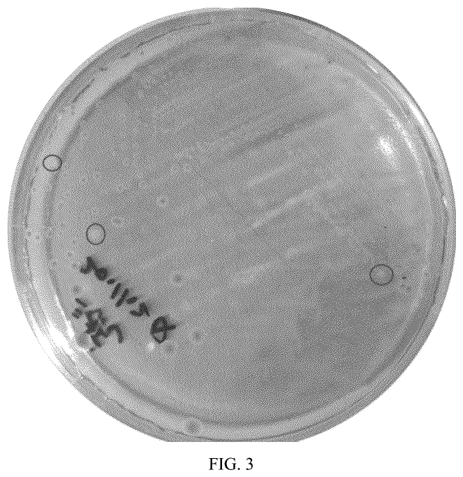

FIG. 4A shows the SDS-PAGE of the effluent of the recombinant protein after passing through Nickel ion affinity chromatography provided with embodiments. The numberals represents the tubes with containing the effluent. D and M refer to dimers and monomers, respectively.

FIG. 4B shows the Western Blotting of the dimer of the recombinant protein provided with embodiments. D refers to dimers.

FIG. 4C shows the Western Blotting of the monomer of the recombinant protein provided with embodiments. M refers to monomers.

FIG. 5 shows the ultraviolet absorption spectrum of the recombinant protein provided with embodiments.

FIG. 6 shows the circular dichroogram of the recombinant protein provided with embodiments.

FIG. 7 shows the three-dimensional structure diagram of the recombinant protein provided with embodiments.

FIG. 8A shows the ultraviolet diagram of the dialysis solution titrated by E2.

FIG. 8B shows the fluorescence diagram of the dialysis solution titrated by E2.

FIG. 8C shows the fluorescence diagram of the GPER protein solution titrated by E2.

FIG. 8D shows the linear fitting diagram of the binding constant of GPER and E2.

FIG. 9 shows the result by Autodock to molecular dock the recombinant protein and E2.

### DETAILED DESCRIPTION OF THE EMBODIMENTS

As used herein, “recombinant protein” generally refers to a novel protein with the same biological function as the wild-type GPER, which is formed at least by the insertion, mutation or deletion of other amino acid sequences.

Embodiments provide a recombinant protein of GPER. The recombinant protein has an amino acid sequence shown as SEQ ID NO:1.

Embodiments disclose a polymer. The polymer includes a plurality of a recombinant protein of GPER. The recombinant protein shows as SEQ ID NO:1. The plurality is selected from 2, 3, 4 or 5.

Embodiments disclose a synthetic DNA. The synthetic DNA codes a recombinant protein of GPER. The recombinant protein shows as SEQ ID NO:1. In some embodiments, the synthetic DNA shows as SEQ ID NO:3.

Embodiments disclose a recombinant plasmid. The recombinant plasmid includes a basal vector and a synthetic DNA. The synthetic DNA codes a recombinant protein of GPER. The recombinant protein shows as SEQ ID NO:1.

In another aspect, embodiments disclose a recombinant host. The recombinant host carries a recombinant plasmid. The recombinant plasmid includes a basal vector and a synthetic DNA. The synthetic DNA codes a recombinant protein of GPER. The recombinant protein shows as SEQ ID NO:1.

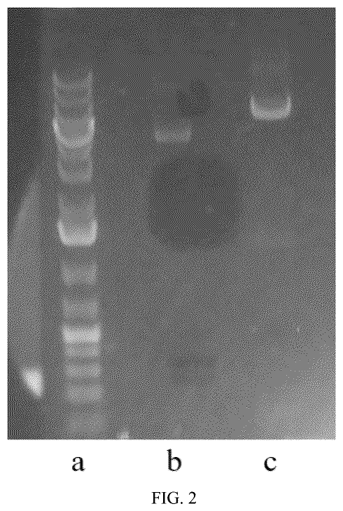

As shown in FIG. 2, the protein samples collected by recombinant expression and purification mainly exist in dimer form. This suggests that these dimers formed by GPER have dimer activation potential, and have unique structural and protein functions, which is crucial for studying the signaling mechanism of GPER receptors in humans and developing agonist drugs that initiate GPER activation patterns.

Embodiments disclose uses of a recombinant protein of GPER. The recombinant protein shows as SEQ ID NO:1. The uses are selected from preparations of antibodies, drug screening kits, western blot kits, ELISA kits, or agonists for initiating the activation of GPER.

In some embodiments, the recombinant protein is used as a target for screening drugs, and prepared to be a reagent of a kit. For example, the kit for screening drugs may include: a standard with containing the recombinant protein, enzyme solution, buffer1 and buffer2. And a process for screening may include: gradient diluting a drug to be tested (e.g. estradiol); incubating the gradient-diluted drug to be tested with the standard at the same concentration for 15-30 min; adding the enzyme solution for 5-10 min incubation; adding the buffer2 after the incubation and boiling for 5 min at 100° C.; and testing samples from the boiling solution by SDS-PAGE and Western blotting. And the buffer1 may used as a blank control.

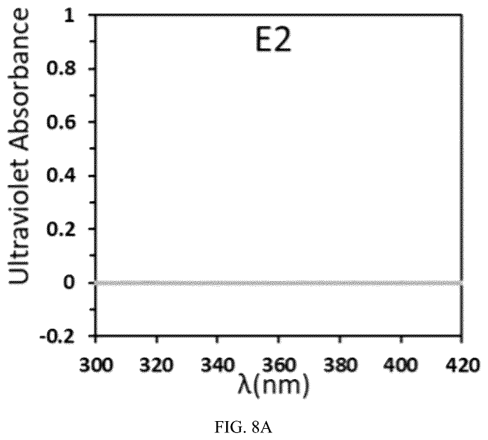

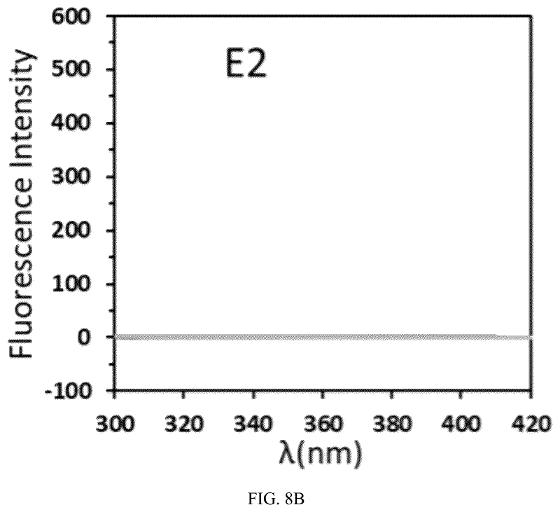

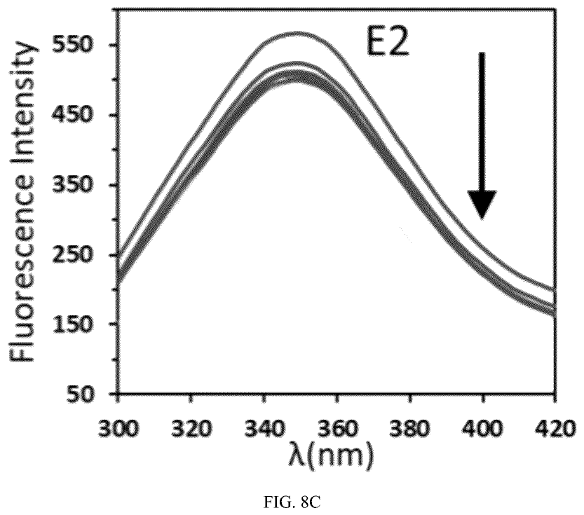

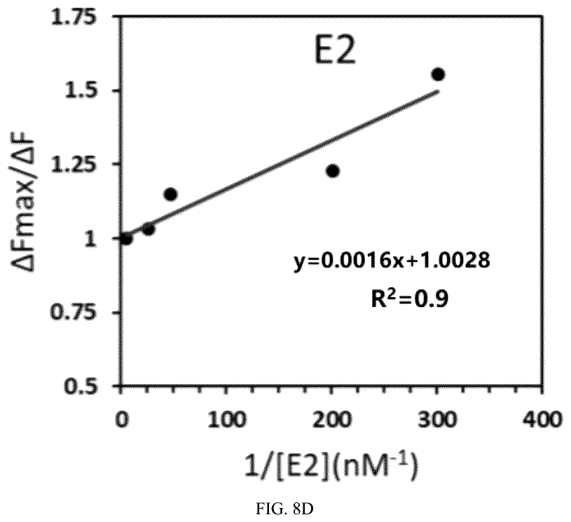

As shown in FIG. 8, the standard has thicker bands than the blank control after incubation with estradiol. These results indicate that the combination with small molecule drugs can enhance the thermodynamic stability and enzymatic hydrolysis resistance of GPER, so as to protect GPER from protease hydrolysis. The results indicate that the estradiol could directly interact with the recombinant protein of GPER and is a potential GPER modulator.

In another aspect, embodiments disclose a synthetic DNA. The synthetic DNA codes the recombinant protein. In some embodiments, the synthetic DNA shows as SEQ ID NO:8. The synthetic DNA could be synthesized by chemically or amplified by PCR.

In another aspect, embodiments disclose a recombinant plasmid. The recombinant plasmid includes a basal vector and the synthetic DNA. The synthetic DNA codes a recombinant protein of GPER. The basal vector may be selected from a group consisting of pDNR, pUC (e.g. pUC19, pUC18), pSTV, pBR (e.g. pBR322), pHSG (e.g. pHSG299, pHSG298, pHSG399, pHSG398), RSF (e.g. RSF1010), pACYC (e.g. pACYC177, pACYC184), pMW (e.g. pMW119, pMW118, pMW219, pMW218, pQE (e.g. pQE30) or their derivatives.

In another aspect, embodiments disclose a recombinant host. The recombinant host includes a host and a recombinant plasmid in the host. The recombinant plasmid includes a basal vector and the synthetic DNA. The synthetic DNA codes a recombinant protein of GPER. The host may be selected from a group consisting of Escherichia (Escherichia coli), Corynebacterium (Corynebacterium glutamicum), Bacillus (Bacillus subtilis), Saccharomyces (Saccharomyces cerevisiae), Pichia (Pichia stipitis), Aspergillus (Aspergillus oryzae), or insect ovarian cells. The host may also be a strain with missing specific genes.

Another aspect, embodiments disclose a method for preparing a recombinant protein. The recombinant protein shows as SEQ ID NO:1. The method includes: synthesizing a first plasmid, the first plasmid includes a first target sequence shown as SEQ ID NO:2, the first target sequence codes GPER; inserting a second target sequence shown as SEQ ID NO:3 into the first plasmid to obtain a second plasmid; culturing a recombinant host, the recombinant host includes a host and the recombinant plasmid; extracting the recombinant protein from the culture.

In some embodiments, a process of synthesizing the first plasmid includes: synthesizing the first target sequence; digesting the first target sequence and pFasBac1; ligating the digested first target sequence and the digested pFasBac1; transferring the ligation product into E. coli; screening positive colonies; and extracting the first plasmid from the positive colonies.

In some embodiments, a process of obtaining the second plasmid includes: synthesizing the second target sequence for coding the recombinant plasmid of GPER; and inserting the second target sequence into the first plasmid.

More specific embodiments of this disclosure are set out below, but do not constitute a restriction on the manner in which this disclosure is implemented.

In some embodiments, the method for preparing the recombinant protein of GPER includes the following steps:

### 1. Construct the First Plasmid

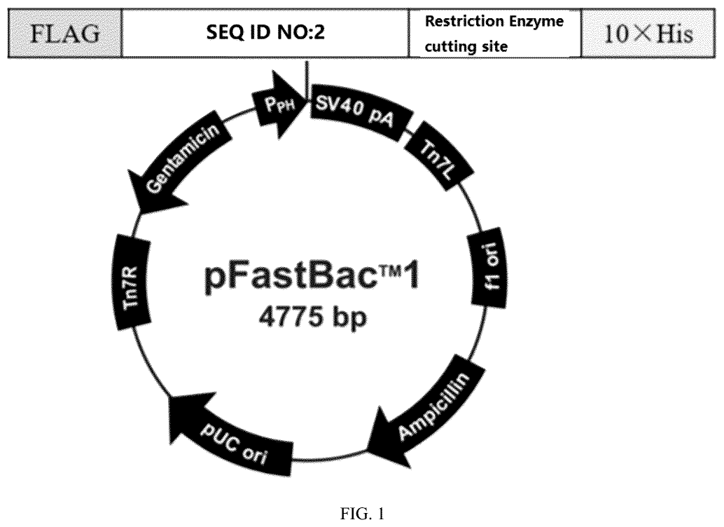

As shown in FIG. 1, the first plasmid was prepared by inserting the first target sequence into pFastBac1(HG-VKI0132, Invitrogen). Therein, the first target sequence shown as SEQ ID NO:2 for coding GPER.

In some embodiments, a PCR of a plasmid for expressing wide type GPER (pDNR-Dual, BC011634, HsCD00005110, ampicillin, 2218 bp, https://dnasu.org) was executed. The product from the PCR and pFastBac1(HG-VKI0132, Invitrogen) were digested, and ligated by T4 DNA ligase. The ligation product was transferred into E. coli. And positive colonies were screened. And the first plasmid could be extracted from the positive colonies.

In some embodiments, a system of the PCR of pDNR-Dual measured in 50 μL included: 1 μL pDNR-Dual, 1 μL forward primer (attggcgcgccgctgtcccacccgctcctg, SEQ ID NO:4), 11 μL reverse primer (attggccggcccggcaaatttgttttctg, SEQ ID NO:5), 4 μL dNTPs with 2.5 mM, 10 μL 5× Buffer, 0.5 μL high-fidelity enzyme, and residual ddH₂O. A procedure of the PCR of pDNR-Dual included: 98° C., 2 min; 98° C., 10 s; 65° C., 30 s; 72° C., 30 s (15-30 s/kb); 35 cycles; 72° C., 10 min; 4° C.

In some embodiments, the PCR product was subjected to gel electrophoresis, and the gel blocks containing the target bands were cut for gel recovery (QIAGEN). The measured concentration of PCR product was 61.2 ng/μL after extraction.

In some embodiments, a system of the digestion for the PCR product of pDNR-Dual measured in 50 μL included: 2 g PCR product, 20 U AscI and 20 U FseI (total 12 μL), 5 μL Cutsmart buffer, residual ddH₂O. A condition of the digestion was at 37° C. for 2 h.

In some embodiments, a system of the digestion for pFasBac1 measured in 50 μL included: 2 g pFasBac1, 2.6 μL AscI (10000 U/mL), 13 μL FseI (10000 U/mL), 6.48 μL Cutsmart buffer, residual ddH₂O. The condition of the digestion was at 37° C. for 2 h.

In some embodiments, a system of the ligation measured in 20 μL included: 2 μL digested pFasBac1, 8 μL digested PCR product of pDNR-Dual, 1 μLT4 DNA ligase (Invitrogen), 2 μL 10× buffer, residual ddH₂O. The condition of the ligation was at room temperature for 20 min.

In some embodiments, a process of transferring the ligation product into E. coli. included: adding 20 μL ligation product into 50 μL competent E. coli Top10 and incubating for 20 min; heat shocking at 42° C. for 60-90 s and incubating at room temperature for 2 min; adding 600 μL LB medium; culturing at 37° C., 200 rpm for 30 min; coating on LB plates containing 25 g/ml ampicillin; and incubating at 37° C. overnight. At last, colonies on the LB plates could be verified by a Colony PCR. A system of the Colony PCR measured in 20 μL included: 2 inoculating loops of colonies, 1 μL forward primers (10 μM, attggcgcgccgctgtcccacccgctcctg, SEQ ID NO:4), 1 μL reverse primers (10 μM, attggccggcccggcaaatttgttttctg, SEQ ID NO:5), 2 μL dNTPs with 2.5 mM, 2 μL 10× Buffer, 0.25 μL Taq DNA Polymerase (5 U/μL), and residual ddH₂O. A procedure of the Colony PCR included: 98° C., 30 s; 98° C., 10 s; 55° C., 90 s; 72° C., 30 s; 25 cycles; 72° C., 10 min; 4° C.

In some embodiments, a process of extracting the first plasmid included: inoculating 100 μL positive colonies into 5 mL LB liquid with containing 25 g/ml ampicillin; and culturing at 37° C., 200 rpm overnight; and extracting 3 mL first plasmid by a plasmid extraction kit (TIANGEN Biotech (Beijing) Co., Ltd.); testing its concentration and sequencing (Beijing Tsingke Biotech Co., Ltd.).

### 2. Construct the Second Plasmid

In some embodiments, a process for constructing the second plasmid included: synthesizing the second target sequence shown as SEQ ID NO:2 for coding the recombinant protein of GPER; and inserting into the first plasmid.

In some embodiments, a PCR with Phusion Plus DNA Polymerase was used to amplify the second target sequence. A system of the PCR measured in 20 μL included: 1 μL second target sequence, 1 μL forward primers (10 μM, atgaagaagtatacctgtaccgtctgcgga, SEQ ID NO:6), 1 μL reverse primers (10 μM, ttcctccacctcttcaaattggtcctt, SEQ ID NO:7), 1.6 μL dNTPs with 2.5 mM, 4 μL 5×GC Buffer, 0.2 μL Phusion Plus DNA Polymerase (Thermo Scientific), and residual ddH₂O.

In some embodiments, a PCR with Phusion Plus DNA Polymerase was used to amplify the first plasmid. A system of the PCR measured in 20 μL included: 1 μL first plasmid, 1 μL forward primers (10 μM, tttgaagaggtggaggaacaccgtgggctgcggc, SEQ ID NO:8), 1 μL reverse primers (10 M, tacaggtatacttcttcatccggtgcgccctgacc, SEQ ID NO:9), 1.6 μL dNTPs with 2.5 mM, 4 μL 5×GC Buffer, 0.2 μL Phusion Plus DNA Polymerase (Thermo Scientific), 0.6 μL DMSO, and residual ddH₂O.

In these embodiments, the procedure of the PCR with Phusion Plus DNA Polymerase included: 98° C., 30 s; 98° C., 10 s (25 cycles); 55° C., 30 s (25 cycles); 72° C., 10 s (25 cycles); 72° C., 10 min; 4° C.

In some embodiments, a process of inserting the second target sequence into the first plasmid included: digesting 20 μL second target sequence and 20 μL first plasmid with 1 μL DpnI (NEC) respectively, and incubating at 37° C. for 1 h; mixing the digested second target sequence and the digested first plasmid with a mole ratio of 1:1 at 4° C.; and adding the mixture into 40 μL competent E. coli Top10 and incubating for 30 min; after heat shocking at 42° C. for 90 s, adding into 350 μL LB medium; culturing at 37° C., 250 rpm for 1 h; centrifuging at 6000 rpm for 2 min; resuspending with 50 μL LB medium; coating on LB plates containing 25 g/ml ampicillin; and incubating at 37° C. overnight. At last, colonies on the LB plates could be verified by a Colony PCR. A system of the Colony PCR measured in 20 μL included: 2 inoculating loops of colonies, 1 μL forward primers (10 μM, attggcgcgccgctgtcccacccgctcctg, SEQ ID NO:4), 1 μL reverse primers (10 μM, attggccggcccggcaaatttgttttctg, SEQ ID NO:5), 2 μL dNTPs with 2.5 mM, 2 μL 10× Buffer, 0.25 μL Taq DNA Polymerase (5 U/μL), and residual ddH₂O. A procedure of the Colony PCR included: 98° C., 30 s; 98° C., 10 s; 55° C., 90 s; 72° C., 30 s; 25 cycles; 72° C., 10 min; 4° C. The second plasmid could be extracted by a plasmid extraction kit (TIANGEN Biotech (Beijing) Co., Ltd.), and its concentration could be tested. The second plasmid could be verified by Sequencing (Beijing Tsingke Biotech Co., Ltd.).

In these embodiments, the process for constructing the second plasmid also included: adding 1 μL (250 ng) the second plasmid into 40 μL competent E. coli DH10Bac(SHBCC D24959, https://b2b.baidu.com/) and incubating for 30 min on ice; after heat shocking at 42° C. for 90 s and incubating for 2 min on ice; adding into 300 μL LB medium; culturing at 37° C., 200 rpm for 4 h; coating 20 μL the culture liquid on LB plates with containing kanamycin, gentamicin, and tetracyclinem that had been coated with X-gal and IPTG beforehand; and incubating at 37° C. for 48 h. At last, white colonies on the LB plates could be shifted into the liquid LB with containing kanamycin, gentamicin, and cultured at 37° C., 200 rpm for 18 h. The second plasmid could be extracted from the thalli of the colonies by a Plasmid extraction kit (Tiangen). The concentration of the second plasmid could be tested by Microvolume UV-Vis Spectrophotometer (Thermo Scientific NanoDrop One).

### 3. Express and Purify the Recombinant Protein

### (1) Prepare the Host

Frozen SF9 cells (insect ovarian cells, Thermo Scientific) for using as the host were rapidly melted at 37° C., and resuspended with ESF-921 medium (Experssion Systems), and shifted into T25 cell culture flasks (Corning), and stationary cultured at 27° C. The host with a concentration of 1×10⁶ cells/mL could be prepared by sub-cultured with a ratio of 1:4 every 2 days.

### (2) Transfect

5 g of the second plasmid was mixed with 250 μL of ESF-921 medium, and 10 μL of transfection reagent was mixed with 250 μL of ESF-921 medium too. The mixture with containing the second plasmid was dropwise added into the mixture with containing the transfection reagent, and incubated at room temperature for 10 min. The incubated mixture was added into 2 mL of the host with a concentration of 1.5×10⁶ cells per well at a 24-well plate, and cultured at 27° C., 300 rpm for 3 days. The supernatant that contained P0 viruses, was gained by centrifuging at 2000 rpm for 15 min.

### (3) Prepare P1 Viruses

400 μL of P0 viruses were mixed into 40 mL host with a concentration of 2×10⁶ cells/mL, and shaking cultured at 27° C., 125 rpm for 48 h. The cell suspension was collected, and centrifuged at 2000 rpm for 15 min. And the recombinant protein consisted in precipitates from the centrifuging. The precipitates were cryopreserved at −80° C.

### (4) Purify the Precipitates

1) The precipitates were melted and re-suspended by low salt buffer on ice. The melt was ground by a homogenizer in ice water, and centrifuged at 40000 rpm, 4° C. for 30 min for a first time. Precipitates from the first time centrifuging were resuspended by low salt buffer and 1 mM PMSF. And the resuspended solution was ground by a homogenizer in ice water, and centrifuged at 40000 rpm, 4° C. for 30 min for a second time. Then, precipitates from the second time centrifuging were resuspended by low salt buffer, and ground by a homogenizer in ice water, and centrifuged at 40000 rpm, 4° C. for 30 min for a third time. Precipitates from the third time centrifuging were resuspended by low salt buffer with containing 15% glycerinum, and cryopreserved at −80° C.

2) The cryopreserved precipitates from step 1) were centrifuged at 14000 rpm, 4° C. for 15 min, and the precipitates were resuspended by buffer, and incubated at 4° C. for 2 h. The incubated solution was mixed with 0.5% DDM and 0.1% CHS, incubated at 4° C. for 2 h, and centrifuged at 14000 rpm, 4° C. for 15 min. The supernatant was mixed with 2 mL Nickel ion affinity chromatography filler, that had been equilibrated by low salt buffer. And the mixture was placed on a rotary mixer at 4° C. overnight.

3) The mixed filler was rinsed by Wash buffer, gradientally eluted by several 10 mL eluents with containing different concentrations of imidazole. The effluents were collected by 1.5 mL per ep tube. And the recombinant protein consisted in the effluent and verified by Coomassie brilliant blue staining.

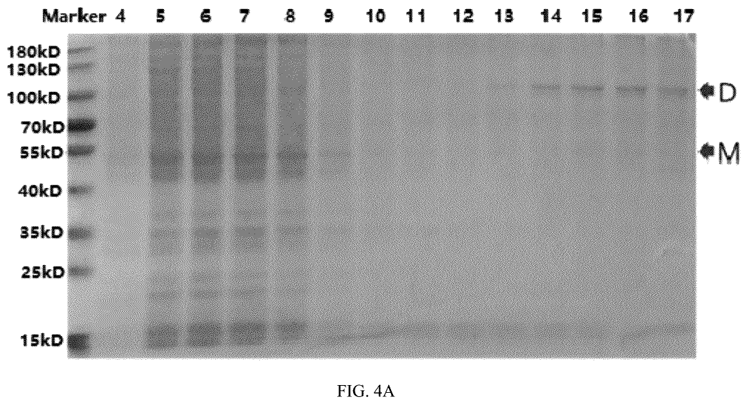

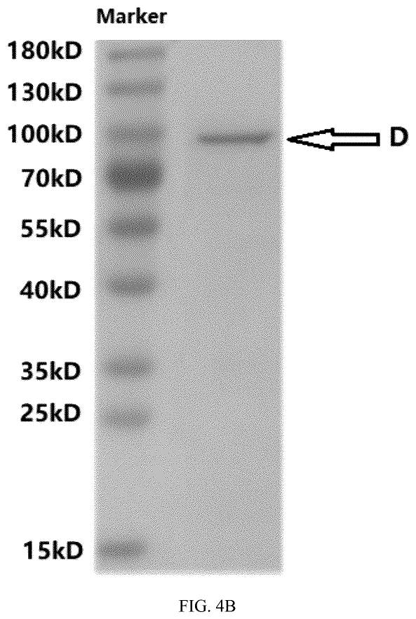

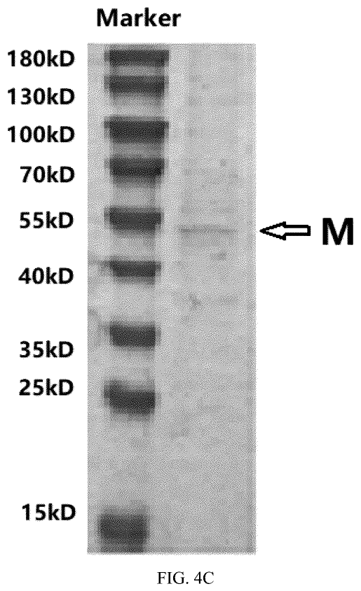

FIG. 4A shows that the recombinant protein consisted in the effluent.

### 4. Western Blot

In a process of Western Blot, Gel block of samples from the Electrophoresis was electro-transferred to NC membrane at 100 V for 90 min. The NC membrane was then washed with TBST for three times, and blocked with 5% skim milk on a shaker for 2 h. After blocking, the NC membrane was then washed with TBST for three times, and incubated with GPER antibody (Invitrogen, PA5-28647) overnight. After that, the NC membrane was then washed with TBST for three times, and incubated with secondary antibody (HRP-labeled Goat Anti-Rabbit IgG, ABclonal, AS014, BR) at room temperature for 3 h, washed with TBST for three times, visualized the result in the dark room with DAB.

As shown in FIG. 4B, the recombinant protein is stably expressed, and the monomer and the dimer of the recombinant protein is also stably expressed.

### 5. Ultraviolet Spectroscopy

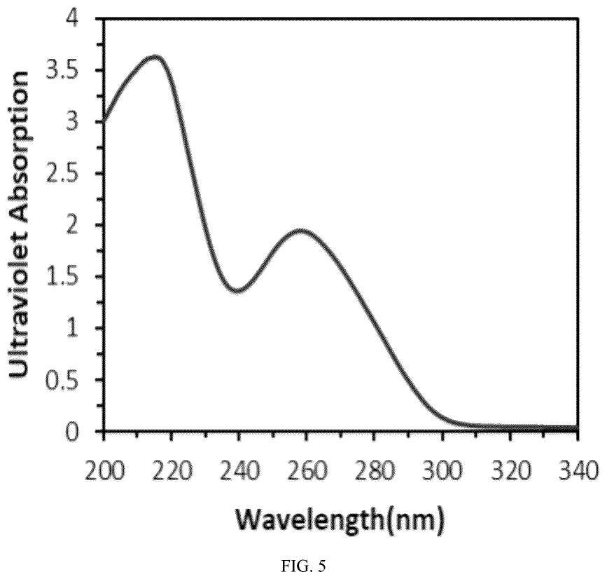

A test result by Ultraviolet spectroscopy as shown in FIG. 5, the recombinant protein has a protein characteristic peak at about 260 nm. The molar extinction coefficient of the recombinant protein could be predicted according to its amino acid sequence, and the molar extinction coefficient is 48970 L·mol⁻¹cm⁻¹. The concentration of recombinant protein in samples prepared by the above action is 1.08 mg/mL.

### 6. Circular Dichroism

Circular dichroism is used to test the secondary structure of the recombinant protein at 180-250 nm.

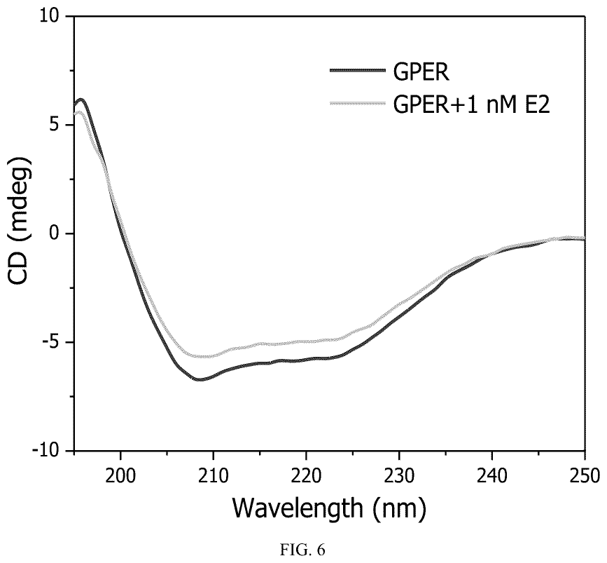

As shown in FIG. 6, the recombinant protein has two negative peaks at 208 nm and 222 nm. This is the circular dichroic signature of the alpha helix. This indicates that the recombinant protein is rich in α-helical secondary structure. After mixing with 1 nM estradiol (E2), the circular dichroism spectrum has changed. This indicates that the recombinant protein could bind to E2, an endogenous ligand of GPER.

### 7. Three-Dimensional Structure

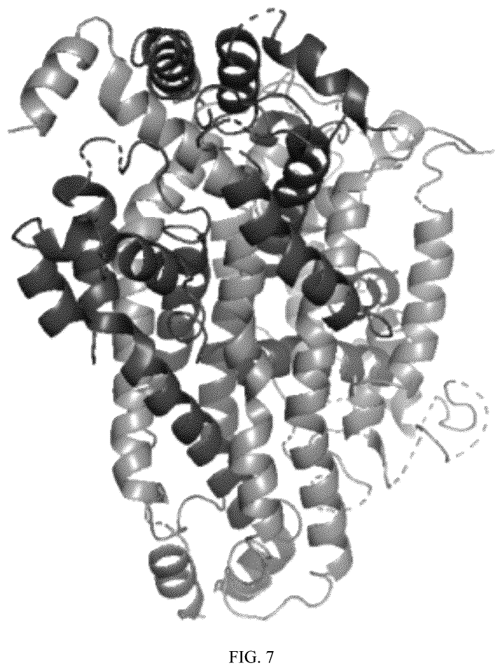

FIG. 7 shows the dimer of the recombinant protein. Thereby, both monomers exhibit the characteristic seven-helical structure of GPCR family proteins. This suggests that the recombinant protein could be used to screen for drugs targeting GPER.

### 8. Fluorescence Titration

The recombinant protein was diluted to a UV absorption value of 0.2 at 280 nm, and titrated with E2. The titrant was scanned by fluorescence for three times, the excitation wavelength was 295 nm, the width of the two slits were set to 7.5 nm, and the emission wavelength was 200-500 nm. According to the change of fluorescence curve, E2 solutions were added to the recombinant protein solutions from low to high concentration. And the recombinant protein solutions without mixed with E2 solutions were used as blank controls.

As shown in FIG. 8C, the peak of endogenous fluorescence intensity of the recombinant protein solutions appeared at about 340 nm. After adding E2 solution at first time, the fluorescence intensity increases and the red shift occurs. With the increase of E2 concentration, the peak value of fluorescence intensity continues to decrease, but the peak position remains unchanged until the peak value does not change (the total volume change caused by controlling the drop does not exceed 5%).

Dialyzates for imidazole removal (20 mM Tris-HCl with 100 mM NaCl) were titrated with E2 solutions as the same manner, as blank controls.

FIG. 8A shows no ultraviolet absorption at 300-420 nm. This precludes the possibility that the change of fluorescence value caused by the change of ultraviolet absorption during E2 titration.

As shown in FIG. 8B, the dialyzates have no fluorescence signal by itself. The fluorescence curve accords with the formula: ΔFₘₐₓ/ΔF=([E2]+K_{d})/[E2]. Therein, ΔF is the change in fluorescence intensity after each drop addition, ΔFₘₐₓ is the change in maximum fluorescence intensity, [E2] is the concentration of E2, and K_{d} is the dissociation constant. A fitting line plot is obtained by taking the reciprocal of E2 concentration as the X-axis and ΔFₘₐₓ/ΔF as the Y-axis. As shown in FIG. 8D, the K_{d} is 0.0016 nM, so the association constant is 6.25×10¹¹ M⁻¹.

### 9. Molecular Docking

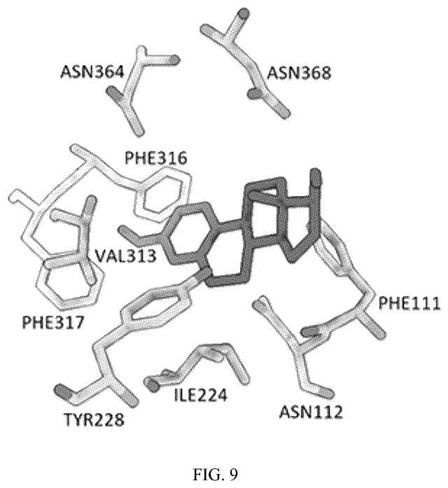

FIG. 9 shows the result by Autodock to molecular dock the recombinant protein and E2. Active residues of the recombinant protein binds to E2 include amino acids at positions 111, 112, 224, 228, 313, 316, 317, 364 and 368.

These results indicate that the recombinant protein could bind to the natural ligand E2 of GPER, and could be used as a test material to carry out GPER drug screening studies including agonists.

The above is only the preferred embodiments of this disclosure and is not intended to limit this disclosure. Any modification, equivalent replacement, improvement, etc., made within the spirit and principle of this disclosure shall be included in the scope of this disclosure.

What is claimed is:

1. A recombinant protein of G protein-coupled estrogen receptor comprising SEQ ID NO:1, whereby said recombinant protein has the binding activity to E2.

2. The recombinant protein according to claim 1, wherein said recombinant protein has amino acid residues binding to E2 at 111, 112, 224, 228, 313, 316, 317, 364 and 368.

3. The recombinant protein according to claim 1, wherein said recombinant protein has a molar extinction coefficient of 48970 L·mol⁻¹cm⁻¹ by the testing result of Ultraviolet spectroscopy.

4. The recombinant protein according to claim 1, wherein said recombinant protein has two negative peaks at 208 nm and 222 nm by the testing result of Circular dichroism.

5. The recombinant protein according to claim 1, wherein said recombinant protein has a seven-helical structure of G protein-coupled estrogen receptor family proteins.

6. A polymer comprising a plurality of a recombinant protein of G protein-coupled estrogen receptor, said recombinant protein comprising SEQ ID NO:1.

7. The polymer according to claim 6, wherein said plurality is selected from 2, 3, 4 or 5.

8. Uses of a recombinant protein of G protein-coupled estrogen receptor, wherein said recombinant protein comprising SEQ ID NO:1, said uses are selected from preparations of antibodies, drug screening kits, Western Blot kits, ELISA kits, or agonists for initiating the activation of G protein-coupled estrogen receptor.
